(19) United States
(12) Patent Application Publication
(10) Pub. No.: US 2020/0223731 A1
(43) Pub. Date: Jul. 16, 2020

(54) Method to Improve the Dewatering of Farm Waste Sludges

(71) Applicants: Jordan PHASEY, Nightcliff, Darwin, Northern Territory (AU); Phinite Inc., Raleigh, NC (US)

(72) Inventor: Jordan Phasey, Nightcliff (AU)

(21) Appl. No.: 16/615,010

(22) PCT Filed: May 14, 2018
(86) PCT No.: PCT/US18/32517

§ 371 Date: Nov. 19, 2019

Related U.S. Application Data
(60) Provisional application No. 62/510,289, filed on May 24, 2017.

(51) Int. Cl. C02F 11/16; C02F 3/28; C02F 3/32; B09B 3/00; C12P 5/02

(52) U.S. Cl. CPC C02F 11/16; C02F 3/28; C02F 2101/16; B09B 3/00; C12P 5/023; C02F 3/327

(57) ABSTRACT

A system for processing a manure sludge includes in a preferred embodiment: (a) an anaerobic digester; (b) a drying bed planted with a plurality of macrophytes, said macrophytes being nurtured so they are growing and engaging in evapotranspiration bed; and (c) a transfer assembly adapted to deliver a manure sludge from the anaerobic digester to the drying bed so that the roots of the plants contact manure sludge transferred to the drying bed so that the sludge is dried by evapotranspiration. The anaerobic digester and transfer assembly are adapted to: (i) treat raw farm waste slurry to provide a biostabilized manure sludge composition; and (ii) deliver the biostabilized manure sludge composition to the drying bed at a solids content of at least about 1% by weight.

### CLAIM FOR PRIORITY

This application is based on U.S. Provisional Application No. 62/510,289 filed 24 May 2017 of the same title, the priority of which is hereby claimed and the disclosure of which is incorporated herein by reference.

### TECHNICAL FIELD

This invention is in the field of farm waste management, and is a process that treats wastes and dewaters the solids, allowing them to be utilized more efficiently.

### BACKGROUND

Farms often yield waste products which are relatively dilute mixtures of farm derived solids and water. Such mixtures often contain manure from animals, waste feed, residuals from mortalities and other detritus which are generated on the farm from time to time. For the purpose of this application, these mixtures are referred to as manure.

Due to the volume and polluting nature of manure, farms use a manure management system. The primary purpose is to contain and store the manure, and some systems are also designed to treat the manure (remove odors, pathogens, nitrogen, phosphorus etc.). Often these systems include a simple solids separation step, such as gravity settling or mechanical separation, to yield a primarily liquid product (called effluent), and a more concentrated solids product (called sludge).

Farmers (the operators of the farms, as defined above) must ensure sludge is removed and disposed of periodically (the frequency is dictated by the manure management system design). The reasons for this are myriad and include but are not limited to: regulatory; aesthetic; environmental; production efficiency etc. This exercise is often costly and difficult to perform.

Sludge contains many elements such as carbon, nitrogen, phosphorus and trace minerals valuable for a variety of purposes. Often the most desirable disposal method is to beneficially reuse nutrients as fertilizer by spreading solids on agricultural land; however other options exist to dispose of or beneficially reuse the solids and their contents. Collectively, these activities are referred to herein as “utilization”.

Of particular concern when considering the utilization of sludges is the low concentration of desirable elements within the sludge (e.g., P<1% w/w to 5% w/w as dry matter), and the moisture content of sludges (<1% to 15% total solids), when they are removed from the manure management system. Unless transport distances to the location of utilization is very short, it is often more economical to concentrate the desirable elements in the sludge prior to utilization. Many options exist to achieve this, such as chemical extraction and precipitation; and dewatering.

This invention aims to improve the shortcomings of conventional dewatering and drying approaches by increasing the solids content and stability (odor generating potential) of the dewatered solid product.

Below is a description of the state of the art in dewatering practice. How these methods work, and their limitations are outlined below:
Drying pans—consist of a layer of drainage media (usually sand and gravel) upon which solids are applied. Water is removed by gravity drainage and evaporation. A maximum thickness of <10 in. is usually employed to maximize thickening of solids, and a final solids content of 12-15% w/w can be achieved. Further concentration is necessary to minimize road transport costs. They are relatively low cost, though are not widely used as rainfall in humid climates makes them useless, and in dry climates evaporation pans are cheaper to construct.
Evaporation pans—are simple basins in which manure is placed. In dry climates water is removed by evaporation and with extended drying and some management (e.g., turning the solids), solids content of 100% can be achieved. They are the lowest cost technology where land availability is not a problem and are extensively used in dry climates. In climates where rainfall exceeds evaporation, they do not work for obvious reasons. Covering them to exclude rainfall is not cost effective as the structures are sometimes required to withstand strong winds (storms and cyclones/hurricanes).

Mechanical and filtration approaches include:
Centrifuges—use the density difference between water and solids to separate solids by spinning the mixture and applying forces many times that of gravity to aid separation of solids. Sometimes a coagulant or flocculant is used to assist dewatering. They are relatively expensive to purchase, complex to operate and yield a solids product from 15-30% w/w usually.
Filter presses—utilize a filter material and a machine to apply pressure and/or vacuum to the filter bag to extract water from solids. Often a coagulant is used to assist dewatering. They are relatively expensive to purchase, complex to operate and yield a solids product from 15-30% w/w usually.
Geotextile bag dewatering—utilize a large geotextile filter bag or tube, solids are pumped into the bag and water is filtered through the bag by gravity over time. Often a coagulant or flocculant is used to assist dewatering. They usually yield a solids product from 12-20% w/w.

Filter presses and centrifuges have seen limited use by farmers, due to cost and operational complexity. Geotextile bag dewatering is the most common approach used in humid climates as rainfall is not a problem and large amounts of solids can be handled in a short amount of time, at relatively low cost and operational simplicity. However the product is only 12-30% solids, or 70-88% moisture. Subsequent land transport for utilization is expensive as the farmer is paying to transport mostly water.

Drying technology is capable of yielding products with a higher solids content, such as:
Heated dryers, flash distillation, and other methods—utilize chemical energy from an external fuel source to remove moisture through a combination of evaporation and boiling. They are expensive to purchase and to operate, due to the large energy requirements to remove large amounts of moisture. Solar dryers use solar heat energy to save on energy costs, but are expensive to construct as they require large amounts of land to achieve practical solids throughput.

Heated dryers have seen extensive use in Europe for farm solids drying where subsidies exist to pay for the process and waste heat from Combined Heat and Power operations. This is not the norm in the rest of the world.

The following references are illustrative of the state of the art.
Licht—U.S. Pat. No. 6,250,237 B1
Geary and Moore (1999) Suitability of a treatment wetland for dairy wastewaters. Water Science and Technology. Vol 40. No 3. Pp 179-185.
Sun, Gray, Biddlestone and Cooper (1999) Treatment of agricultural wastewater in a combined total flow-downflow reed bed system. Water Science and Technology. Vol 30. No 3. Pp 139-146.
Karpiscak, Freitas, Gerba, Sanchez and Shamir (1999) Management of dairy waste in the sonoran desert using constructed wetland technology. Water Science and Technology. Vol 30. No 3. Pp 57-65.
Hunt and Poach (2001) State of the art for animal wastewater treatment in constructed wetlands. Water Science and Technology. Vol 44. No 11-12. Pp 19-25.
Harrington and McInnes (2009) Integrated constructed wetlands (ICW) for livestock wastewater management. Bioresource Technology. Vol 100. Pp 5498-5505.
Meers, Tack, Tolpe and Michels (2008) Application of a full-scale constructed wetland for tertiary treatment of piggery manure: monitoring results. Vol 193. Pp 15-24.
Kantawanichkul and Somprasert (2005) Using a compact combined constructed wetland system to treat agricultural wastewater with high nitrogen. Water Science and Technology. Vol 51. No 9. Pp 47-53.
Nielsen (2005) Sludge reed bed facilities: operations and problems. Water Science and Technology. Vol 51. No 9. Pp 99-107.
Heinss and Kootatep (1998) Use of reed beds for faecal sludge dewatering.
Vazquez, Varga, Plana and Soto (2013) Vertical flow constructed wetland treating high strength wastewater from swine slurry composting. Ecological Engineering. Vol 50. Pp 37-43.
Summerfeldt, Adler, Glenn and Kretschmann (1999) Aquaculture sludge removal and stabilization within created wetlands. Aquacultural engineering. Vol 19. Pp 81-92.
Edwards, Gray, Cooper, Biddelstone and Willoughby (2001) Reed bed dewatering of agricultural sludges and slurries. Water Science and Technology. Vol 44. 11-12. Pp 551-558.
Wallace—U.S. Pat. No. 6,200,469 B1

Other references of general interest include the following patents and published applications. U.S. Pat. No. 7,718,063 (2010) to Jacquet, entitled “Treating Pollutants By Phytoleaching” discloses a method of treated polluted wastewater, air or soil by treatment with a planted filter bed operated with aerobic and anaerobic periods with acidification to a pH of 4 to 7. U.S. Pat. No. 7,951,296 (2011) to Williams, entitled “Apparatus and Method for Agricultural Animal Wastewater Treatment” discloses a wastewater treatment method and apparatus. The system employs an initial anaerobic wastewater treatment followed by batch floating aquatic plant treatment. U.S. Pat. No. 6,846,343 (2005) to Sower, entitled “Fertilizer Manufacture From Animal Wastes and Method of Producing Same” discloses fertilizer made from animal wastes. The fertilizer is typically produced by pressing, drying and optionally pelletizing the material. U.S. Pat. No. 6,112,458 to Pabsch (2000), entitled “Processing of Sewage Sludge Into Humus” discloses processing sewage sludge, including drying the sludge with fast-growing plants. United States Patent Application Publication No. US 2017/0260073 (2017), entitled “Sludge Treatment System With Sludge Drying Acceleration Function” discloses using hydroponic plants for accelerating drying of reservoir silt. United States Patent Application Publication No. US 2016/0297699 (2016), entitled “Psychrophilic Anaerobic Digestion of Ammonia-Rich Waste” discloses a method of treating ammonia containing manure with low temperatures in an anaerobic digester with psychrophilic bacteria. United States Patent Application Publication No. US 2006/0024282 (2006), entitled “Biological Fertilizer Compositions Comprising Swine Manure: discloses preparing manure for fertilizer using manure dried to a moisture content of less than or equal to 5%. See ¶¶[0229]-[0232]. United States Patent Application Publication No. US 2005/0145552, entitled “Animal Waste Effluent Treatment” discloses a method of treating animal waste effluent, including ultrasonic treatment and use of flocculants and zeolites. Still further references of general interest include U.S. Pat. No. 6,409,788; United States Patent application Publication No. 20090250393A1; U.S. Pat. No. 5,690,827; Chinese Patent CN103723892B; Chinese Patent CN203639301; European Patent No. EP1812350A2; Chinese Patent CN201010606Y; Behrends, L., et al. (2003), Reciprocating Constructed Wetlands for Treating High Strength Anaerobic Lagoon Wastewater, Animal, Agricultural and Food Processing Wastes-IX (p. 1), American Society of Agricultural and Biological Engineers; Behrends, L. and Bock, B. (2000) Reciprocating Constructed Wetlands (ReCip) for Treating Anaerobic Lagoon Wastewater; Hunt, P. G., Stone, K. C., Matheny, T. A., Poach, M. E., Vanotti, M. B., & Ducey, T. F. (2009), Denitrification of nitrified and non-nitrified swine lagoon wastewater in the suspended sludge layer of treatment wetlands, Ecological engineering, 35(10), 1514-1522; Steinmann, C. R., Weinhart, S., & Melzer, A. (2003), A combined system of lagoon and constructed wetland for an effective wastewater treatment, Water Research, 37(9), 2035-2042; Ramachandra, T. V., Bhat, S., & Vinay, S. (2017), Constructed Wetlands for Tertiary treatment of Wastewater. ENVIS Technical Report 124, Energy & Wetlands Research Group, CES, Indian Institute of Science, Bangalore 560012; and Shappell, N. W., Billey, L. O., Forbes, D., Matheny, T. A., Poach, M. E., Reddy, G. B., & Hunt, P. G. (2007), Estrogenic activity and steroid hormones in swine wastewater through a lagoon constructed-wetland system, Environmental science & technology, 41(2), 444-450.

The prior art fails to disclose a waste treatment where a pre-treatment step is employed on high strength waste in order to remove ammonia and BOD from manure solids prior to the use of plants to dewater the solids.

The prior art related to wetlands can generally be summarized into two categories:
1. Agricultural applications of horizontal flow or vertical flow wetlands to treat the effluent, i.e. dilute wastewater, from an agricultural application, with the exception of Summerfelt et al. (1999), noted above, and;
2. Municipal applications of sludge treatment wetlands; treating water treatment sludge, primary sludge, waste activated and fecal sludge of human origin.

Several companies may be considered to be skilled in the art of using wetlands to treat sludge: Agricultural Requisites and Mechanizations Limited (ARM—http://armreedbeds.co.uk/) in Staffordshire, UK; Orbicon (Steen Nielsen the main person—https://www.orbicon.com) a consulting engineering company in Denmark; Naturally Wallace (Scott Wallace—http://naturallywallace.com) a consulting engineering company in Minnesota.

ARM in particular have a strong history in agricultural engineering. A review of their website includes the following statement (Retrieved on 26 Apr. 2017):

“Finding Agriculture solutions to run-off from farmyards and housed livestock was our principal focus when we first started to design and construct reed beds in the 1980's. Although initial results indicated that agricultural wastewater containing swine and dairy effluent were too strong for traditional passive reed beds, new advances in wetland technology and the introduction of Forced Bed Aeration™ has provided a natural solution to these high strength effluents. The installation of a reed bed system for the treatment of contaminated water on a farm can bring particular ecological benefits, are cost-effective and a long-term solution to a long-term problem.”

The ARM website then outlines one example, using a vertical flow wetland to treat wastewater from a vegetable processing facility. The strength issue stated by the ARM website refers to a well-known issue with the use of plants in wastewater and sludge management—that high loadings of biodegradable matter (measured by the Biochemical Oxygen Demand—BOD) leads to anaerobic conditions, which kill plants that require oxygen to function; and also causes conversion of nitrogen containing species to ammonia, which is toxic to plants. This has prevented their widespread application in the field of agricultural waste management—despite their other benefits such as simple construction and operation and low cost.

In addition, while accidental and opportunistic plant growth has been observed on sludge removed from treatment processes (e.g., Anaerobic lagoon sludge), the author is aware of no examples where plants have been used to remove moisture from treated sludge (as defined above) in a designed, managed and successful way. Examples of design would include the addition of sludge to an engineered structure, containing plants for the purpose of dewatering. Examples of management would include the deliberate use (planting of, or presence of a species which is particularly self-propagating) of plants for the express purpose of removing moisture from the solids (opportunistic plant growth would not remove sufficient moisture to be practically useful in this context). An example of the successful use of plants would be performing the above activity to achieve a desirable outcome; such as the removal of moisture from, and stabilization (reduction in odor generating potential) of solids derived from farm activities.

### SUMMARY OF INVENTION

There is provided in accordance with the present invention a method and system for processing raw farm waste including manure. Processing includes pre-treating raw farm waste slurry including manure and water to remove nitrogenous waste and biodegradable material in the raw manure such that the treated waste slurry is substantially free of toxic levels of nitrogenous compounds such as ammonia and ammonia-generating compounds and is substantially free of intolerable levels of oxygen-depleting biodegradable waste to provide a biostabilized manure sludge composition; optionally dewatering the biostabilized manure sludge composition to remove free water from the sludge; providing a drying bed planted with a plurality of macrophytes and nurturing the plants so they are growing and engaging in evapotranspiration, said drying bed having a water-impermeable liner and drainage effective to remove free water applied to the drying bed; applying the biostabilized manure sludge composition to the drying bed so that the plant roots to come into contact with the biostabilized manure solids; and through evapotranspiration, drying the biostabilized manure solids to a moisture content of less than about 70% by weight.

Further details and advantages of the invention will become apparent from the discussion which follows.

### BRIEF DESCRIPTION OF DRAWINGS

The invention is described in detail below with reference to the drawings wherein like numerals designate similar features and parts and wherein:

FIG. 1 is a process flow diagram illustrating a manure treatment process in accordance with the present invention;

FIG. 2 is a schematic diagram illustrating a constructed wetland drying bed useful in connection with the present invention;

FIG. 3 is a schematic diagram which represents a planted geotextile tube useful in dewatering sludge in connection with the present invention;

FIG. 4 is a schematic diagram which represents a planted geotextile tube with contained structures to assist in plant growth;

FIG. 5 is a schematic plan view of a system for practicing the present invention;

FIG. 6 is a schematic view in section and elevation of the plant drying bed of the system of FIG. 5;

FIG. 7 is a detail of the construction of the plant drying bed of FIGS. 5 and 6 showing incorporation of a spray system to remove ammonia while applying the sludge to the wetland; and

FIG. 8 is a schematic diagram of yet another system for producing the present invention using an enclosed complete mix anaerobic digester to biostabilize farm waste.

### DETAILED DESCRIPTION

The invention is described in detail below in connection with the Figures for purposes of illustration, only. The invention is defined in the appended claims. Terminology used herein is given its ordinary meaning; for instance, g refers to grams, mg refers to milligrams, m² refers to square meters and so forth. Unless otherwise indicated, %, percent, % (w/w) and the like refers to weight percent. Further definitions are noted below.

Ammonia content is measured by way of Apha (1999) Method 4500-NH₃ or equivalent.

An anaerobic digester includes any suitable apparatus carrying out anaerobic digestion, i.e. hydrolysis, acidogenesis, acetogenesis and methanogenesis of sludge. Typical constructions include continuous or batch wet or dry systems such as plug flow, mixed digesters and uncovered or covered lagoons.

Biochemical Oxygen Demand (BOD) is the amount of dissolved oxygen necessary to break down organic material present in a given aqueous sample. 5 day BOD or BOD(5) is expressed herein in milligrams of oxygen consumed per liter of sample during 5 days of incubation at 20° C., that is, ppm w/v. Suitable test methods include Standard Method 5210B: BOD: 5-Day Test and related procedures such as volatile solids Standard Method 1684 which in agricultural applications correlates to BOD.

Dewatering refers to removing free water from the sludge, that is, water that is not adsorbed on the particle surfaces. Drying, on the other hand refers to removing free water and adsorbed water to achieve higher solids content.

The hydraulic retention time (HRT), also known as hydraulic residence time or t (tau), is a measure of the average length of time that a compound (ex. water) remains in a storage unit (ex. lake, pond, ocean). Hydraulic retention time is the volume of the storage unit divided by the influent flowrate, often expressed in days.

For the purpose of this application, farms include all agricultural activities that have the purpose of producing animals and animal products (for example, but not limited to: Pigs, cattle, dairies, goats, sheep etc.) for subsequent use or consumption.

A “geotextile” bag is a highly porous tubular structure made of porous polymer fabric, typically polypropylene fibers in the form of a woven or a nonwoven fabric.

A macrophyte is an aquatic plant that grows in or near water and is either emergent, submergent, or floating, and includes helophytes (a plant that grows in marsh, partly submerged in water, so that it regrows from buds below the water surface). Examples include Equisetum fluviatile, Glyceria maxima, Hippuris vulgaris, Sagittaria, Carex, Schoenoplectus, Sparganium, Acorus, yellow flag (Iris pseudacorus), Typha and Phragmites australi as well as the various species identified hereinafter.

Unless otherwise indicated “ppm w/v” refers to parts per million concentration weight per volume (w/v), that is, mg component/liter of sludge slurry or effluent. As used herein, “sludge”, “sludge slurry”, “manure slurry” and the like refers to aqueous farm waste, typically manure having a relatively high solids content as compared to the wastewater effluent from a lagoon after solids settling. Typically, sludge refers to aqueous compositions with at least 1 weight percent solids content or more, suitably from 3 weight percent to 5 weight percent solids or more. Solids content is dictated by conditions within the lagoon and can increase with depth up to about 20% solids in some cases. Effluent wastewater from a waste processing lagoon or other wastewater solids removal apparatus has a solids content of less than 1%, typically about 0.5% for lagoon wastewater.

A biostabilized manure sludge composition exhibits a reduced level of ammonia and reduced Bod (5) levels as compared to untreated sludge of the same material and is substantially free of toxic levels of nitrogenous compounds such as ammonia and ammonia-generating compounds as well as being substantially free of intolerable levels of oxygen-depleting biodegradable waste so that the macrophytes thrive after the biostabilized sludge is applied to the drying bed. That is, the sludge is biostabilized when the ammonia content and oxygen-depleting biodegradable waste levels are below toxic levels and growth inhibiting levels to the plants in the drying bed so that the macrophytes thrive and dry the sludge through evapotranspiration. Threshold levels may depend on somewhat on design and selection of the macrophytes. Failure to thrive may be ascertained by observing a reduced growth rate relative to a control and failure to dry the sludge at the designed loading rates. Alternatively, failure to thrive is characterized by a reduction in permeabilty of the sludge layer as evidenced by standing water on the surface for five days. Substantial freedom from toxic ammonia levels may be achieved anywhere below about 1200 ppm w/v ammonia and substantial freedom from intolerable levels of oxygen-depleting biodegradable waste may be achieved at BOD(5) levels of less than 4500 ppm w/v. A treated manure sludge may be considered biostabilized for present purposes when the treated sludge exhibits a reduced level of ammonia and reduced Bod (5) levels as compared to untreated sludge of the same material and solids content obtained by decanting untreated feed to the system. Preferably relative reductions of 25%, 40% or more as compared to the untreated feed are seen in ammonia content and BOD(5) levels. Reductions of 50%, 60%, 70%, 80% or more in ammonia content and BOD(5) levels are even more desirable in some cases.

A fundamental principle employed in this invention is that plants can be used to dry agricultural manure sludge, removing moisture to a point where it can be more economically transported for land application or further processed to prior to use.

This invention is not specific on the species of plant used. Some plants may be better than others (e.g., higher evapotranspiration (ET) rates, better tolerance to ammonia and salt etc.), however the invention is claimed to cover the use of any species of plant to dewater agricultural manure sludge. Likewise, the invention is not specific to apparatus for bringing the plants in contact with the sludge.

In addition, the manure is pre-treated prior to dewatering in order to remove factors such as ammonia-generating nitrogenous matter and matter that leads to Biochemical Oxygen Demand (BOD), which consumes oxygen and inhibits the growth of plants.

Any suitable method may be used to treat the manure prior to dewatering, including technologies now established and yet to be developed treatments.

Specifically, the treatment step removes ammonia below levels which are toxic to whatever species of plant is used to remove the moisture.

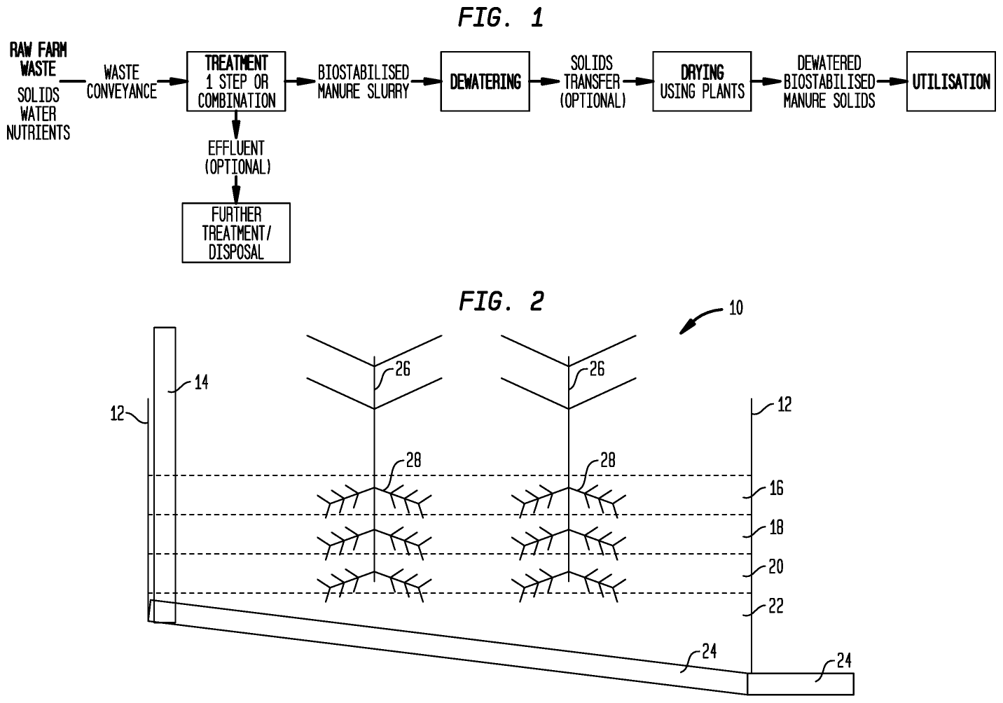

The treatment step may also remove biodegradable matter from the solids. This invention receives raw manure, and generally employs the following process areas to manage manure; a treatment step; one or more dewatering steps; optional further processing step/s; and a utilization step (FIG. 1). The treatment step might be one or more steps such as anaerobic digestion and nitrogen removal which occurs concurrently in an uncovered anaerobic lagoon, or may occur separately with an anaerobic digester or covered anaerobic lagoon and a separate nitrogen removal step or may be carried out with a nitrogen removal process physically disposed within the digester or covered anaerobic lagoon. Likewise, dewatering and drying may be carried out in multiple steps or concurrently as discussed herein.

In one embodiment of the present invention, open, that is uncovered anaerobic lagoons are used in the treatment step to treat manure.

In another embodiment, other techniques are employed in the treatment step to lower ammonia levels in the manure and facilitate plant growth, such as but not limited to; ammonia stripping, nitrification with or without denitrification, ion exchange, electro-dialysis reversal, membrane removal. Such treatment steps may also be used to treat the sludge from a covered anaerobic lagoon or anaerobic digester.

In one embodiment of the present invention, the dewatering step is achieved by applying treated manure to a planted sludge drying bed, as illustrated in FIG. 2.

In another embodiment of the present invention, the dewatering step is achieved through the application of sludge to geotextile membranes with or without polymer addition; followed by the use of plants to progress drying to a relatively high solids contents (40-80% or more); prior to the utilization step.

In another embodiment, the sludge may be dewatered by any other technology such as, but not limited to belt filter press, centrifuge, sand drying bed. Plants may then be used to dry sludge as described herein.

The present invention is directed generally to a system for treating farm waste sludge, which reduces the moisture content of the solids, allowing for more efficient utilization of the solids. Moisture is removed from the solids through evapotranspiration by the growth of plants within the sludge solids. Plant survival, normally a problem with growing plants on high strength wastes, is achieved through substantial reductions in the ammonia and BOD content of the solids through one, or a combination of pre-treatment steps.

An example of one preferred pre-treatment step is the use of an anaerobic lagoon to contain and treat farm waste. Farm waste manure usually contains 1000-2000 ppm ammonia, plus 2000-5000 ppm organic nitrogen, which is converted to ammonia by biological breakdown. Different manure has different levels. A primary application of the present invention is to treat and dry Dairy and Swine manure. Biodegradeable matter is substantially removed from the solids by microbial anaerobic digestion of the material, the carbonaceous matter is converted to methane, which is lost to the atmosphere and inorganic carbon (carbonate species) which remain in the effluent from the lagoon. Biodegradable nitrogenous material is bioconverted to ammonia in the anaerobic digestion process and enters the effluent of the lagoon. Ammonia in the lagoon effluent and sludge is lost to the atmosphere by volatilization in the case of an uncovered anaerobic lagoon. Solids in the waste settle by sedimentation to the bottom of the lagoon.

This anaerobic lagoon process leads to an effluent substantially reduced in solids, BOD, phosphorus and ammonia. The process also leads to a thickened sludge product substantially reduced in volume, BOD, odor generation potential, biodegradable nitrogen and ammonia; and substantially increased in solids content and phosphorus content.

Preferably, the pre-treated form waste or manure sludge has less than 1000 pm ammonia and less than 4000 ppm w/v (BOD5).

The sludge from the anaerobic lagoon is transferred to a planted sludge drying bed where moisture is removed from the sludge by drainage through the bed and evapotranspiration by the plants, leading to a sludge product that is substantially reduced in moisture and therefore weight. This reduction in moisture content makes the material more cost efficient to transport. In addition, flammable material from the plants may lead to a final sludge-plant material that can burn without additional energy input, allowing further concentration of the valuable material within the sludge.

Plant growth within the sludge is facilitated through the removal of deleterious ammonia from the sludge material. This ammonia is toxic to plants.

Drying beds of vertical flow design can tolerate higher ammonia levels than other types of constructed wetlands, due to the intermittent application of moisture, which leads to regular introduction of oxygen into the root and sludge structure. This allows nitrification to take place.

The invention is better understood by reference to the Figures. In FIG. 1 there is provided a process flow diagram wherein farm waste is pretreated to remove organic waste and ammonia to provide a biostabilized manure slurry which is optionally further dewatered before drying a planted bed. The dried biostabilized manure slurry may then be used as fertilizer, for example.

Various features of the system are illustrated in FIGS. 2-4. FIG. 2 is a schematic diagram of a planted sludge drying bed 10 provided with a liner 12, an optional aeration pipe 14, a plurality of layers 16, 18, 20 and 22, wherein layer 16 is a sludge layer, layer 18 is an optional layer of plant growth media (soil, humous, compost, potting soil or mulch), layer 20 is an optional layer of sand and layer 22 is an optional layer of gravel. A drainage pipe is indicated by the reference numeral 24. Plants are indicated at 26 with roots 28.

Through evapotranspiration, sludge layer 16 is dried to a moisture content of less than 70% by weight.

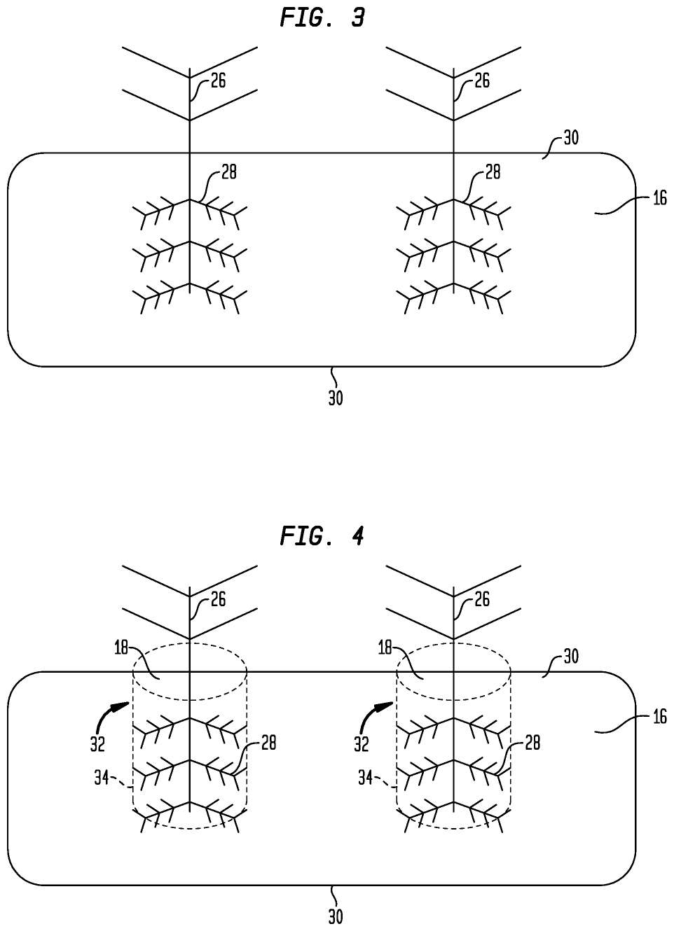

FIG. 3 illustrates schematically a geotextile tube 30 containing sludge 16 planted with plants 26 with roots 28. This configuration may be used in connection with drying the sludge with or without various additional features shown in FIG. 2.

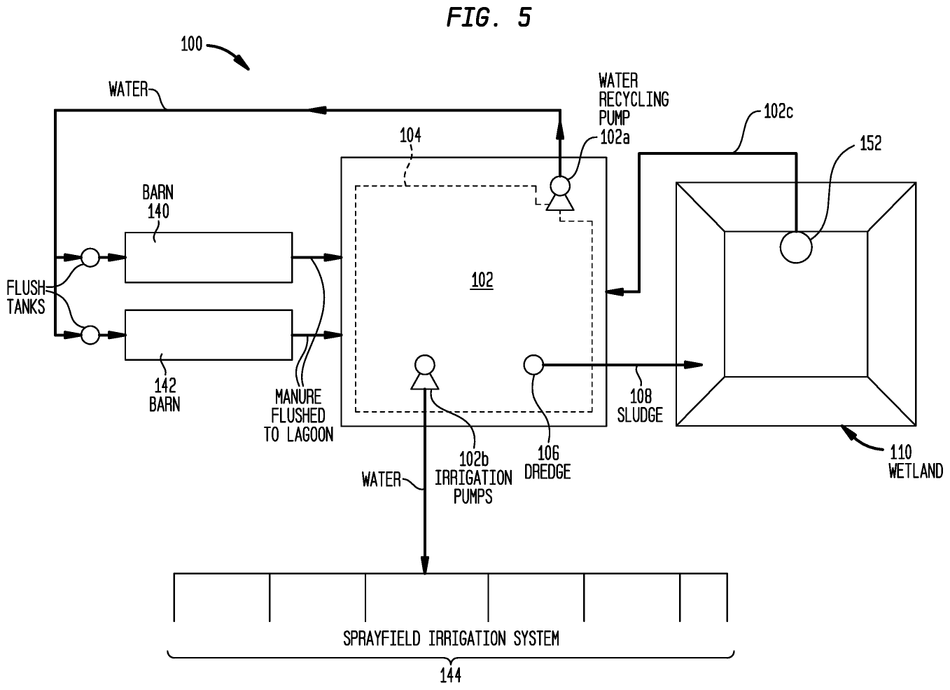

FIG. 4 illustrates a geotextile tube 30 as shown in FIG. 3 with plants 26 having roots 28 and sludge 16. Here, there are provided containment structures 32 in the form of perforated pipes 34 having a plant growth media 18 such as soil, humus, mulch, compost, potting mix and the like.

The planted drying beds of FIGS. 3 and 4 may optionally be used without geotextile tube 30 on an open pile/layer of sludge, if so desired; preferably with drainage underneath the sludge.

A preferred implementation of the present invention includes anaerobic digestion of manure followed by evapotranspiration of the sludge obtained from the digestor in a planted drying bed. Anaerobic digestion leads to the breakdown of carbohydrates and fats into more simple molecules which are then broken down into CO₂ and methane. Proteins (and other digestible nitrogen containing material) are also broken down and due to the anaerobic conditions nitrogen ends up as NH₃ which is water soluble, so ammonia ends up in the water. Anaerobic lagoons may be sized to have a hydraulic retention time (HRT) of about half a year (180 days), so water stays in the system a long time. In an uncovered or open lagoon, the water is in contact with the atmosphere. Ammonia is volatile so it volatilizes to the atmosphere and is lost from the system and thus is removed from the sludge.

In covered anaerobic lagoons and anaerobic digesters, the cover (or enclosure) prevents loss of ammonia (and methane) to the atmosphere; thus higher levels of ammonia are present in the sludge; which may or may not require an ammonia removal step prior to transpiration drying.

Anaerobic digesters, such as enclosed mixed anaerobic digesters are usually designed with a HRT of 30-60 days (smaller to reduce construction cost). The same conversion of protein to ammonia occurs but because they are enclosed the ammonia is not lost to the atmosphere.

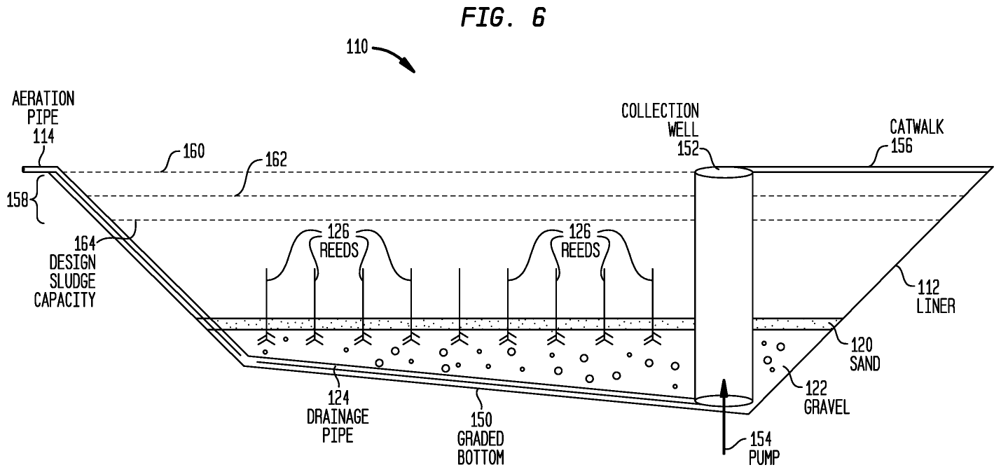

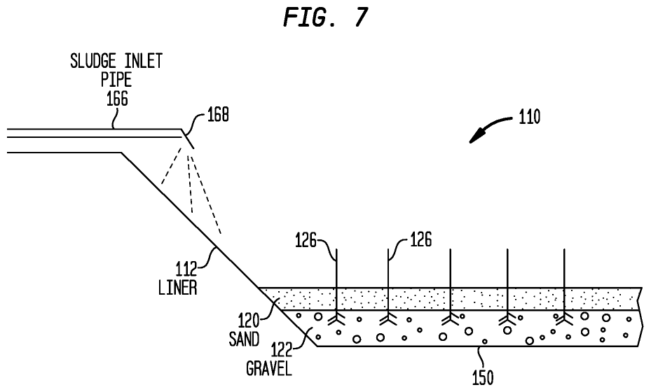

One preferred system 100 is shown in FIG. 5. System 100 includes an anaerobic lagoon 102 which may be of uncovered design or with an optional cover for biogas collection such as floating cover 104 adjacent a planted drying bed which is a vertical flow constructed wetland indicated generally at 110. In lagoon 102 biodegradable organic matter is converted to methane and carbon dioxide which is largely lost to the atmosphere through the lagoon surface in the case of an uncovered lagoon; nitrogenous material is remineralized to ammonia which is also lost to the atmosphere through the lagoon surface, and gravity separation of solids occurs. The farm waste is thus biostabilzed in the lagoon to lower ammonia content and BOD. The solids that settle in the lagoon form a biostabilized sludge layer. Within this layer is disposed a dredge 106, which transfers biostabilized sludge slurry 108 from the bottom of the lagoon through means of a pump and pipe to wetland 110. The vertical flow constructed wetland 110 includes an earthen structure, a liner made with water-impermeable material and a bottom structure that is graded to allow fluid flow as indicated in FIG. 6, discussed hereinafter.

In operation, system 100 recirculates water by way of a pump 102a from lagoon 102 to flush tanks which are used to flush manure from barns 140, 142 to lagoon 102. In the lagoon, solids are separated by gravity and provided to drying bed 110 as sludge slurry 108 after an appropriate HRT in the lagoon by way of dredge 106. Effluent wastewater is also provided from the lagoon to a sprayfield irrigation system 144 by way of an irrigation pump 102b. A collection well 152 provided with a pump 154 (FIG. 6) recirculates wastewater collected from the drying bed to lagoon 102 by way of a line 102c.

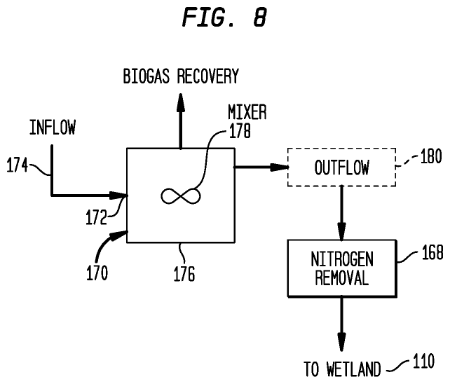

Referring to FIGS. 6, 7 there are shown details of constructed wetland 110 which may be used in connection with any embodiment. Wetland 110 includes a liner 112 and is provided with a graded bottom 150, as well as aeration pipes such as pipe 114 and drainage pipes such as pipe 124, a collection well 152 provided with a pump 154 and a catwalk 156 for servicing the well 152. Also provided at the bottom of wetland 110 are a sand layer 120 and a gravel layer 122.

Wetland 110 is sized to provide a freeboard space 158 to prevent overflow of the wetland including, for example, a first freeboard space 160 and a second freeboard space 162 above a design sludge capacity level 164. Spaces 160, 162 are sized to each accommodate precipitation from a 25 year, 24 hour storm which depends upon the locality.

In cases where a covered anaerobic lagoon or an enclosed complete mix anaerobic digester are used, wetland 110 is provided with a sludge inlet system, including one or more pipes 166 provided with spray nozzles indicated at 168. The spray nozzles act to remove nitrogen from the sludge, that is, operate as a nitrogen removal system which may be required, depending upon ammonia levels. As noted above, alternative nitrogen removal processes can be used such as but not limited to: ammonia stripping, nitrification with or without denitrification, ion exchange, electro-dialysis reversal and membrane removal.

Within the sand and gravel filter media plants 126 are placed; usually reeds or other species tolerant of low oxygen, submerged, brackish and high ammonia conditions. Above the sand is an optional layer of compost or other organic material to facilitate plant establishment and aid filtration of the sludge material. See FIGS. 1,4. Sludge from the anaerobic lagoon is applied to the top of the vertical flow constructed wetland, water within the slurry passes through the layers and collects in the drain pipes, flows to the collection well where pump 154 returns the water to the lagoon or for land application. Solid material in the sludge accumulates above the filter bed. Over time the plants grow their roots through the sludge material and after repeated applications the depth of the material increases. Periodically the sludge material and reed stems are mechanically removed from the system, while leaving a small amount of sludge above the level of the filter sand. After removal the plants recommence growth, sludge application resumes and the system enters operation again for another cycle. The rate of loading of solids in terms of mass of dry matter per unit area of wetland is important to ensuring appropriate performance. Preferred loading rates are enumerated hereinafter. The drainage and aeration pipes allow air to enter the bottom of the wetland, aiding survival of the reeds.

When using a covered anaerobic lagoon to biostabilize the waste, biodegradable organic matter is converted to methane and carbon dioxide, which are collected by a cover such as cover 104 (FIG. 5). Nitrogenous material is remineralised to ammonia, some volatilises through the lagoon surface, however most remains dissolved within the liquid in the lagoon. Gravity separation of solids occurs, forming a sludge layer which is periodically conveyed by means of a pump to wetland 110. Sludge slurry is removed from the lagoon and passes through the nitrogen removal system in the form of nozzles 168 (FIG. 7) prior to entering wetland 110. Through contact with the air, ammonia is volatilized from the sludge material prior to entering the wetland.

Instead of a lagoon, an enclosed anaerobic complete mix digester 170, as shown in FIG. 8, may be used to provide a biostabilized manure slurry to a planted drying bed such as wetland 110. Digester 170 includes an inlet 172 for a manure flow 174 which may be dewatered to slurry form prior to feeding digester 170, as well as an enclosure 176 for collecting biogas with a mixer 178 to uniformly disperse the solids. After an appropriate HRT in digester 170, the biostabilized manure slurry is forwarded to optional further dewatering indicated at 180 followed by nitrogen removal prior to application of the slurry to wetland 110. Nitrogen removal may be carried as shown and described above in connection with FIG. 7 or by nitrogen removal by the other methods noted herein which may occur within enclosure 176 or separately. In wetland 110 the sludge is dried to a moisture content of less than about 70% prior to removing the sludge from the drying bed for further utilization as fertilizer, for example.

### Embodiments of the Invention

Following are illustrative embodiments of the present invention.

Embodiment No. 1 which is a method of processing raw farm waste including manure comprising:
pre-treating raw farm waste slurry including manure and water to remove nitrogenous waste and biodegradable material in the raw manure such that the treated waste slurry is substantially free of toxic nitrogenous compounds such as ammonia and ammonia-generating compounds and is substantially free of oxygen-depleting biodegradable waste to provide a biostabilized manure slurry composition;
providing a dewatering mechanism suitable for removing free water from biostabilized manure solids, and applying the biostabilized manure slurry to this dewatering process;
planting a plurality of plants and nurturing the plants so they are growing and engaging in evapotranspiration;
allowing the plant roots to come into contact with the dewatered biostabilized manure solids; and
through evapotranspiration, drying the biostabilized manure solids to a moisture content of less than about 70% by weight.

Embodiment No. 2 is the method of processing raw farm waste according to Embodiment No. 1, wherein the biostabilized manure slurry composition has an ammonia content of less than 1000 ppm weight per volume (w/v).

Embodiment No. 3 is the method of processing raw farm waste according to Embodiment No. 2, wherein the biostabilized manure slurry composition has an ammonia content of less than 500 ppm w/v.

Embodiment No. 4 is the method of processing raw farm waste according to Embodiment No. 3, wherein the biostabilized manure slurry composition has an ammonia content of less than 300 ppm w/v.

Embodiment No. 5 is the method of processing raw farm waste according to Embodiment No. 4, wherein the biostabilized manure slurry composition has an ammonia content of less than 100 ppm w/v.

Embodiment No. 6 is the method of processing raw farm waste according to any of the foregoing embodiments, wherein the biostabilized manure solids are dried to a moisture content of less than about 60% by weight.

Embodiment No. 7 is the method of processing raw farm waste according to Embodiment No. 6, wherein the biostabilized manure solids are dried to a moisture content of less than about 50% by weight.

Embodiment No. 8 is the method of processing raw farm waste according to Embodiment No. 6, wherein the biostabilized manure solids are dried to a moisture content of less than about 40% by weight.

Embodiment No. 9 is the method of processing raw farm waste according to Embodiment No. 6, wherein the biostabilized manure solids are dried to a moisture content of less than about 30% by weight.

Embodiment No. 10 is the method of processing raw farm waste according to Embodiment No. 6, wherein the biostabilized manure solids are dried to a moisture content of less than about 25% by weight.

Embodiment No. 11 is the method of processing raw farm waste according to Embodiment No. 6, wherein the biostabilized manure solids are dried to a moisture content of less than about 20% by weight.

Embodiment No. 12 is the method of processing raw farm waste according to any of the foregoing embodiments, wherein any species of plant is used.

Embodiment No. 13 is the method of processing raw farm waste according to Embodiment No. 12, wherein the plants comprise macrophytes.

Embodiment No. 14 is the method of processing raw farm waste according to Embodiment No. 12, wherein the plants comprise local species, proven to survive within the conditions delivered by the pre-treatment process.

Embodiment No. 15 is the method of processing raw farm waste according to Embodiment No. 12, wherein the plants belong to the genus Typha.

Embodiment No. 16 is the method of processing raw farm waste according to Embodiment No. 12, wherein the plants are the species Typha augustifolia.

Embodiment No. 17 is the method of processing raw farm waste according to Embodiment No. 12, wherein the plants are the species Typha latafolia.

Embodiment No. 18 is the method of processing raw farm waste according to Embodiment No. 12, wherein the plants are the species Typha domingensis.

Embodiment No. 19 is the method of processing raw farm waste according to Embodiment No. 12, wherein the plants belong to the genus Phragmites.

Embodiment No. 20 is the method of processing raw farm waste according to Embodiment No. 12, wherein the plants are the species Phragmites australis.

Embodiment No. 21 is the method of processing raw farm waste according to Embodiment No. 12, wherein the plants belong to the genus Scirpus.

Embodiment No. 22 is the method of processing raw farm waste according to Embodiment No. 12, wherein the plants are the species Scirpus californicus.

Embodiment No. 23 is the method of processing raw farm waste according to Embodiment No. 12, wherein the plants are the species Scirpus validus.

Embodiment No. 24 is the method of processing raw farm waste according to Embodiment No. 12, wherein the plants belong to the genus Juncus.

Embodiment No. 25 is the method of processing raw farm waste according to Embodiment No. 12, wherein the plants are the species Juncus effusus.

Embodiment No. 26 is the method of processing raw farm waste according to Embodiment No. 12, wherein the plants belong to the genus Schoenoplectus.

Embodiment No. 27 is the method of processing raw farm waste according to Embodiment No. 12, wherein the plants are the species Cyperus papyrus.

Embodiment No. 28 is the method of processing raw farm waste according to Embodiment No. 12, wherein the plants belong to the genus Echinochloa.

Embodiment No. 29 is the method of processing raw farm waste according to Embodiment No. 12, wherein the plants are the species Echinochloa pyramidalis.

Embodiment No. 30 is the method of processing raw farm waste according to any one of the foregoing embodiments, wherein a combination of plant species are used.

Embodiment No. 31 is the method of processing raw farm waste according to any one of the foregoing embodiments, wherein the biostabilized manure slurry composition does not generate substantial additional odors through anaerobic decomposition after nitrogen removal.

Embodiment No. 32 is the method of processing raw farm waste according to Embodiment No. 31, wherein the biostabilized manure slurry composition has a 5 day Biochemical Oxygen Demand of less than 4000 ppm w/v.

Embodiment No. 33 is the method of processing raw farm waste according to Embodiment No. 31, wherein the biostabilized manure slurry composition has a 5 day Biochemical Oxygen Demand of less than 2000 ppm w/v.

Embodiment No. 34 is the method of processing raw farm waste according to Embodiment No. 31, wherein the biostabilized manure slurry composition has a 5 day Biochemical Oxygen Demand of less than 1000 ppm w/v.

Embodiment No. 35 is the method of processing raw farm waste according to any of the foregoing embodiments, wherein the step of pre-treating the raw farm waste slurry is effective to remove at least 50% by weight of the ammonia present therefrom.

Embodiment No. 36 is the method of processing raw farm waste according to Embodiment No. 35, wherein the step of pre-treating the raw farm waste slurry is effective to remove at least 75% by weight of the ammonia present therefrom.

Embodiment No. 37 is the method of processing raw farm waste according to Embodiment No. 35, wherein the step of pre-treating the raw farm waste slurry is effective to remove at least 90% by weight of the ammonia present therefrom.

Embodiment No. 38 is the method of processing raw farm waste according to Embodiment No. 35, wherein the step of pre-treating the raw farm waste slurry is effective to remove at least 95% by weight of the ammonia present therefrom.

Embodiment No. 39 is the method of processing raw farm waste according to any of the foregoing embodiments, wherein the biostabilized manure slurry composition is manure sludge from an anaerobic lagoon.

Embodiment No. 40 is the method of processing raw farm waste according to Embodiment No. 49, wherein the biostabilized manure slurry composition is manure sludge from an anaerobic lagoon which is derived from Dairy manure, Swine manure or combinations thereof.

Embodiment No. 41 is the method of processing raw farm waste according to any of Embodiment Nos. 1 through 38, wherein the biostabilized manure slurry composition is derived from an anaerobic digester.

Embodiment No. 42 is the method of processing raw farm waste according to any one of the foregoing embodiments, wherein the biostabilized manure slurry is treated with ammonia stripping to further remove ammonia.

Embodiment No. 43 is the method of processing raw farm waste according to any one of the foregoing embodiments, wherein the biostabilized manure slurry is treated with nitrification to remove ammonia.

Embodiment No. 44 is the method of processing raw farm waste according to any one of the foregoing embodiments, wherein the biostabilized manure slurry is treated with ion exchange to remove ammonia.

Embodiment No. 45 is the method of processing raw farm waste according to any one of the foregoing embodiments, wherein the biostabilized manure slurry is treated with ammonia removal through gas-permeable membranes.

Embodiment No. 46 is the method of processing raw farm waste according to any one of the foregoing embodiments, wherein the biostabilized manure slurry is treated with electro-dialysis reversal to remove ammonia.

Embodiment No. 47 is the method of processing raw farm waste according to any one of the foregoing embodiments, wherein the biostabilized manure slurry is treated with any other ammonia removal technology, currently available or yet to be invented to further remove ammonia.

Embodiment No. 48 is the method of processing raw farm waste according to any one of the foregoing embodiments, wherein the biostabilized manure slurry is treated with a combination of technologies in order to remove ammonia.

Embodiment No. 49 is the method of processing raw farm waste according to any one of the foregoing embodiments, wherein the biostabilized manure slurry is dewatered and dried within a planted sludge drying bed.

Embodiment No. 50 is the method of processing raw farm waste according to Embodiment No. 49, wherein the solids loading rate of the planted sludge drying bed is less than 500 kg/m²/year.

Embodiment No. 51 is the method of processing raw farm waste according to Embodiment No. 49, wherein the solids loading rate of the planted sludge drying bed is less than 400 kg/m²/year.

Embodiment No. 52 is the method of processing raw farm waste according to Embodiment No. 49, wherein the solids loading rate of the planted sludge drying bed is less than 300 kg/m²/year.

Embodiment No. 53 is the method of processing raw farm waste according to Embodiment No. 49, wherein the solids loading rate of the planted sludge drying bed is less than 200 kg/m²/year.

Embodiment No. 54 is the method of processing raw farm waste according to Embodiment No. 49, wherein the solids loading rate of the planted sludge drying bed is less than 100 kg/m²/year.

Embodiment No. 55 is the method of processing raw farm waste according to Embodiment No. 49, wherein the solids loading rate of the planted sludge drying bed is less than 50 kg/m²/year.

Embodiment No. 56 is the method of processing raw farm waste according to Embodiment Nos. 1-48, wherein the biostabilized manure slurry is dewatered using geo-textile membranes.

Embodiment No. 57 is the method of processing raw farm waste according to Embodiment Nos., wherein the biostabilized manure slurry is dewatered using a filter press.

Embodiment No. 58 is the method of processing raw farm waste according to Embodiment Nos., wherein the biostabilized manure slurry is dewatered using a centrifuge.

Embodiment No. 59 is the method of processing raw farm waste according to Embodiment Nos., wherein the biostabilized manure slurry is dewatered through any dewatering process, currently available or yet to be invented.

Embodiment No. 60 is the method of processing raw farm waste according to any of the foregoing embodiments, wherein plant growth is encouraged through the use of growth media such as potting mix, to minimize the proximity of young plant roots to sludge during the establishment phase.

Embodiment No. 61 is the method of processing raw farm waste according to Embodiment No., wherein the plant growth media contains facility for passive aeration of the plant roots.

While the invention has been described in detail, modifications within the spirit and scope of the invention will be readily apparent to those of skill in the art. Such modifications are also to be considered as part of the present invention. In view of the foregoing discussion, relevant knowledge in the art and references discussed above in connection with the foregoing description and Background of the Invention, the disclosures of which are all incorporated herein by reference, further description is deemed unnecessary. In addition, it should be understood from the foregoing discussion that aspects of the invention and portions of various embodiments may be combined or interchanged either in whole or in part. Furthermore, those of ordinary skill in the art will appreciate that the foregoing description is by way of example only, and is not intended to limit the invention.

1. A method of processing raw farm waste including manure comprising:
pre-treating raw farm waste slurry including manure and water to remove nitrogenous waste and biodegradable material in the raw manure to provide a biostabilized manure sludge composition having a solids content of at least 1 weight percent solids;
optionally dewatering the biostabilized manure sludge composition to remove free water from the sludge;
providing a drying bed planted with a plurality of macrophytes and nurturing the plants so they are growing and engaging in evapotranspiration, said drying bed having a water-impermeable liner and drainage effective to remove free water applied to the drying bed;
applying the biostabilized manure sludge composition having a solids content of at least 1 weight percent solids to the drying bed so that the plant roots to come into contact with the biostabilized manure solids; and
through evapotranspiration, drying the biostabilized manure solids to a moisture content of less than about 70% by weight.

2. The method according to claim 1, wherein the biostabilized manure sludge composition has an ammonia content of less than 1000 ppm weight per volume (w/v) prior to application to the drying bed.

3. The method according to claim 2, wherein the biostabilized manure slurry composition has an ammonia content of less than 500 ppm w/v prior to application to the drying bed.

4. The method according to claim 1, wherein the biostabilized manure sludge is dried to a moisture content of less than about 40% by weight.

5. The method according to claim 1, wherein the macrophytes belong to the genuses Typha, Phragmites, Scirpus, Juncus, Schoenoplectus, or Echinochloa.

6. The method according to claim 1, wherein the macrophytes comprise the species Cyperus papyrus.

7. The method of claim 1, wherein the biostabilized manure sludge composition has a 5 day Biochemical Oxygen Demand of less than 4000 ppm w/v prior to application to the drying bed.

8. The method of claim 7, wherein the biostabilized manure sludge composition has a 5 day Biochemical Oxygen Demand of less than 2000 ppm w/v prior to application to the drying bed.

9. The method of claim 8, wherein the biostabilized manure slurry composition has a 5 day Biochemical Oxygen Demand of less than 1000 ppm w/v prior to application to the drying bed.

10. The method of claim 1, wherein the step of pre-treating the raw farm waste slurry is effective to remove at least 75% by weight of the ammonia present therefrom.

11. The method of claim 1, wherein the biostabilized manure sludge composition is manure slurry from an uncovered anaerobic lagoon.

12. The method of claim 11, wherein the biostabilized manure sludge composition is manure sludge from an uncovered anaerobic lagoon which is derived from Dairy manure, Swine manure or combinations thereof.

13. The method of claim 1, wherein the biostabilized manure sludge composition is derived from a covered anaerobic lagoon or an enclosed mixed anaerobic digester.

14. The method according to claim 1, wherein the farm waste is treated to remove ammonia therefrom by way of a methodology selected from: ammonia stripping; nitrification; treatment with an ion-exchange resin; treatment with gas-permeable membranes; and electro-dialysis reversal.

15. The method of claim 1, wherein the solids loading rate to drying bed is less than 500 kg/m²/year.

16. The method of claim 15, wherein the solids loading rate of the planted sludge drying bed is less than 100 kg/m²/year.

17. A system for processing raw farm waste comprising:
(a) an anaerobic digester;
(b) a drying bed planted with a plurality of macrophytes, said macrophytes being nurtured so they are growing and engaging in evapotranspiration, said drying bed having a water-impermeable liner and drainage effective to remove free water applied to the drying bed;
(c) a transfer assembly adapted to deliver a manure sludge from the anaerobic digester to the drying bed so that the roots of the plants contact manure sludge transferred to the drying bed so that the sludge is dried by evapotranspiration.
wherein the anaerobic digester and transfer assembly operate to: (i) treat raw farm waste slurry, including manure and water to remove nitrogenous waste and biodegradable material to provide a biostabilized manure sludge composition; and (ii) deliver the biostabilized manure sludge composition to the drying bed at a solids content of at least about 1% by weight.

18. The system according to claim 17, wherein the anaerobic digester is an uncovered anaerobic lagoon.

19. The system according to claim 17, wherein the anaerobic digester is selected from covered anaerobic lagoons and enclosed mixed anaerobic digesters.

20. The system according to claim 19, wherein the transfer assembly has a plurality of nozzles through which the manure sludge is sprayed for purposes of removing ammonia from the sludge.
